(19) United States
(12) Patent Application Publication
(10) Pub. No.: US 2004/0136956 A1
(43) Pub. Date: Jul. 15, 2004

(54) Asialo-interferons and the treatment of liver cancer

(76) Inventors: Nicholas P. Barker, Southborough, MA (US); Daniel K. Podolsky, Wellesley, MA (US)

Correspondence Address: CLARK & ELBING LLP, 101 FEDERAL STREET, BOSTON, MA, 02110, US

(21) Appl. No.: 10/654,763

(22) Filed: Sep. 3, 2003

Related U.S. Application Data
(60) Provisional application No. 60/408,265, filed on Sep. 5, 2002.

(51) Int. Cl. A61K 38/21

(52) U.S. Cl. 424/85.5; 424/85.6; 424/85.7

(57) ABSTRACT

This invention features methods for preparing and using asialo-interferons, including asialo-interferon-α, asialo-interferon-α2a, asialo-interferon-α2b, asialo-interferon-β, asialo-interferon-β1a, asialo-interferon-β1b and asialo-interferon-γ, for treating liver cancer. Asialo-interferon therapy may be used alone or in combination with other antineoplastic therapies.

### CROSS-REFERENCE TO RELATED APPLICATIONS

This application claims benefit of the filing date of the co-pending U.S. Provisional Application No. 60/408,265 (filed Sep. 5, 2002), hereby incorporated by reference.

### FIELD OF THE INVENTION

This invention relates to the treatment of liver cancer.

### BACKGROUND OF THE INVENTION

Primary liver cancer occurs when abnormal liver cells undergo uncontrolled growth. In contrast to many other types of cancer, the number of people who develop and die from liver cancer is increasing. Many patients with chronic liver diseases, including cirrhosis and hepatitis, are at increased risk for developing liver cancer. In the United States, the incidence of primary liver cancer increased 71 percent between 1975 and 1995, and the number of patients diagnosed with liver cancer each year continues to rise. In 2002, the American Cancer Society estimates that 16,600 new cases of primary liver cancer and bile duct cancer will be diagnosed in the United States, and that 14,100 Americans will die from the disease.

The most common form of primary liver cancer in both children and adults is hepatocellular carcinoma, accounting for 80 to 90 percent of all liver cancers. Several distinct clinical types of hepatocellular carcinoma occur, including diffuse-type hepatocellular carcinoma, febrile-type hepatocellular carcinoma, and cholestatic hepatocellular carcinoma. Hepatoblastoma is another form of liver cancer that is relatively rare and most often affects young children.

The prognosis in most cases of liver cancer is poor. Current therapies offer limited effectiveness for treating liver cancer. While interferon has been used successfully for the treatment of other types of cancer, such as hairy cell leukemia, chronic myelogenous leukemia, and melanoma, solid tumors of the liver have been less susceptible to treatment with interferon, possibly due to interferons' rapid clearance from the blood. Additionally, interferon treatment often causes adverse side effects and toxicity at dosage levels required for cancer therapy; thus, a need exists for developing therapeutic agents that prevent or treat liver cancer.

### SUMMARY OF THE INVENTION

In one aspect, the invention features a method for treating a patient having liver cancer by administering an effective amount of a pharmaceutical composition containing a mammalian asialo-interferon. In preferred embodiments, the liver cancer expresses an asialo-glycoprotein receptor. In a most preferred embodiment, the liver over-expresses an asialo-glycoprotein receptor.

In another aspect, the invention features a method for treating a patient having a liver cancer that expresses an asialo-glycoprotein receptor by: (a) testing the liver cancer for expression of an asialo-glycoprotein receptor, and (b) administering to the patient an effective amount of a composition containing a mammalian asialo-interferon. In one embodiment, testing of the liver cancer is performed on a tissue sample obtained from the patient by biopsy. In another embodiment, the asialo-glycoprotein receptor is overexpressed and testing of the liver cancer is performed using a non-invasive imaging technique.

Liver cancers amenable to treatment using either of the foregoing methods include, for example, diffuse-type hepatocellular carcinoma, febrile-type hepatocellular carcinoma, and cholestatic hepatocellular carcinoma, hepatoblastoma, hepatoid adenocarcinoma, and focal nodular hyperplasia. In preferred embodiments of these methods, the asialo-interferon in a human asialo-interferon. Suitable asialo-interferons include asialo-interferon-α, -β, and -γ.

In other embodiments, the methods further contain a second anti-neoplastic therapy. Suitable anti-neoplastic therapies include, for example, surgical intervention (i.e., tumor resection), chemotherapy, and radiation therapy.

The therapeutic methods of the invention may also be used to treat metastatic liver cancer. Metastatic liver cancers amenable to treatment include, for example, metastatic prostate cancer, metastatic colorectal cancer, metastatic breast cancer, metastatic lung cancer, metastatic pancreatic cancer, metastatic melanoma, and metastatic leukemias and lymphomas.

By “interferon” is meant the family of highly homologous species-specific proteins known as interferons, that inhibit viral replication and cellular proliferation and modulate immune response and are substantially identical to interferon-α, -β, or -γ, or biologically active fragments thereof. Methods for evaluating the biological activity of interferon are widely known (e.g., Monkarsh et al., Anal. Biochem. 247:434-440, 1997; Grace et al., J. Interferon Cytokine Res. 21: 1103-1115, 2001; Bailon et al., Bioconj. Chem. 12: 195-202, 2001; Pepinsky et al., J. Pharmacol. Exp. Therap. 297:1059-66, 2001). Human interferons are grouped into three classes based on their cellular origin and molecular structure: interferon-α (leukocytes), interferon-β (fibroblasts), and interferon-γ (lymphocytes).

By “interferon-α” is meant a protein containing an amino acid sequence that is substantially identical to the interferon-α2 mature polypeptide (amino acids 24-188 of Accession No:P01563; SEQ ID NO:1), or a biologically active fragment thereof. Thus, interferon-α includes the interferon-α2 precursor polypeptide (Accession No:P01563; SEQ ID NO:1) and fragments that retain the biological activity of mature interferon-α (e.g., anti-proliferative activity). Also included in this definition are the variant forms of interferon-α2 including, for example, interferon-α2b (R46K mutation of SEQ ID NO:1) and interferon-α2c (R57H mutation of SEQ ID NO:1). Interferon-α2b is an O-linked glycoprotein. Interferon-α14c is a N-linked glycoprotein that is glycosylated at Asn-72. Natural interferon is commercially available under the name of Wellferon (Glaxo-SmithKline), Alferon (Interferon), Sumiferon (Sumitomo) and Multiferon (Viragen). Non-glycosylated interferon-α is also commercially available including, for example, recombinant interferon-α2a, under the name Roferon®-A (Roche), recombinant interferon-α2b, under the name Intron®-A (Schering Plough), and recombinant interferon-α2c, under the name of Berofor alpha 2 (Boehringer Ingelheim). Recombinant consensus interferon-con 1 is available under the name of Infergen (Amgen). Of course, prior to use in the composition and methods of this invention, any non-glycosylated interferon must be glycosylated with an oligosaccharide having a terminal galactose residue.

By “interferon-β” is meant a protein containing an amino acid sequence that is substantially identical to the mature interferon-β polypeptide (amino acids 22-187 of Accession No:P0574; SEQ ID NO:2), or a biologically active fragment thereof. Thus, interferon-β includes, in addition to the mature interferon-β protein that does not contain the signal peptide, the interferon-β precursor polypeptide (Accession No:P01574; SEQ ID NO:2) that does contain the signal peptide, and fragments thereof having the biological activity of interferon-β (e.g., anti-proliferative activity). Interferon-β is a glycoprotein that is glycosylated at Asn80 of the mature interferon-β protein. Recombinant forms of interferon-β have been developed and are commercially available. Interferon-β1a is available under the name Avonex® (Biogen) and Rebif® (Serono). Interferon-β1b is available under the name of Betaseron (Berlex).

By “interferon-γ” is meant a protein containing an amino acid sequence that is substantially identical to the mature interferon-γ polypeptide (amino acids 21-166 of Accession number P01579; SEQ ID NO:3), or a biologically active fragment thereof. Thus, interferon-γ proteins include, in addition to the mature interferon-γ polypeptide that does not contain the signal peptide, the interferon-γ precursor protein (Accession number P01579; SEQ ID NO:3) that contains the signal peptide, and fragments thereof having the biological activity of interferon-γ (e.g., antiproliferative activity). Interferon-γ is glycosylated at Asn48 and, in the dimer, at Asn120. Interferon-γ is commercially available under the name Actimmune® (InterMune).

By “asialo-interferon” is meant a glycosylated interferon lacking a terminal sialic group that is present in the native glycosylated interferon. Removal of the terminal sialic acid residue exposes the underlying galactose moiety. It is the terminal galactose that is recognized by the asialoglycoprotein receptor. Preferably, asialo-interferon contains at least 50%, 70%, 80%, 90%, or even 95% of the carbohydrate moieties present in the native interferon. Most preferably, asialo-interferon lacks only the terminal sialic acid residue. Asialo-interferons can be produced by removing one or more sialic acid groups from a glycosylated interferon, such as interferon-α, -β, or -γ. This removal may be accomplished, for example, by mild acid hydrolysis, or treatment of native glycosylated interferon, such as interferon-α, -β, or -γ, with purified neuroaminidase. For interferons containing more than one sugar chain, selective desialylation may be accomplished using specific neuroaminidase (sialidase) enzymes. Specifically excluded by this definition are completely deglycosylated interferons, including interferons that are typically produced by prokaryotic cells and interferons produced by eukaryotic cells and enzymatically or chemically deglycosylated. Of course, because the goal of removing the sialic acid residue is to create a glycosylated interferon having at least one terminal galactose residue on an oligosaccharide chain, a terminal galactose residue may be engineered by any other appropriate means including, for example, covalently attaching an oligosaccharide to a deglycosylated interferon.

By “antineoplastic therapy” is meant any medical procedure or treatment used to inhibit, partially or completely, the proliferation of a neoplasm. Typically, antineoplastic therapies include surgical procedures that remove some or all of the neoplastic cells from the patient (e.g., hepatectomy), radiation therapy, and chemotherapy. Particularly useful classes of antineoplastic chemotherapeutics that can be administered in combination with the asialo-interferons according to the present invention include, for example, alkylating agents, antimetabolites, nitrosoureas, and plant alkaloids.

By “liver cancer” is meant any disorder in which tissues or cells (e.g., hepatocytes) of the liver undergo abnormal uncontrolled proliferation. Liver cancers include, but are not limited to, hepatocellular carcinoma, such as diffuse-type hepatocellular carcinoma, febrile-type hepatocellular carcinoma, and cholestatic hepatocellular carcinoma, hepatoblastoma, hepatoid adenocarcinoma, and focal nodular hyperplasia.

Patients whose liver cancer expresses the asialo-glycoprotein receptor are amenable to treatment with asialo-interferon; these patients may be identified using diagnostic methods that are standard in the art (e.g., Burgess et al., Hepatology 15:702-706, 1992; Hirose et al., Biochem. and Biophys. Research Comm. 287:675-681, 2001; Hyodo et al., Liver 13:80-5, 1993; Trere et al., Br. J. Cancer 81:404-8, 1999).

By “asialo-glycoprotein receptor-expressing liver cancer” is meant any liver cancer that contains neoplastic cells expressing detectable levels of the asialo-glycoprotein receptor protein (Accession No.:NP_{—}001662 or P07307) or functionally equivalent protein. The neoplastic liver cells may be assessed for asialo-glycoprotein receptor expression using any appropriate in vivo, ex vivo, or in vitro technique. For example, cells extracted from a patient during a biopsy or surgical resection can be characterized for asialo-glycoprotein receptor expression using standard immunohistochemistry, Northern or Western blotting techniques, or an ELISA. Asialo-glycoprotein receptors are known to the skilled artisan (e.g., Spiess et al, Proc. Natl. Acad. Sci. 82:6465-6469, 1985; Spiess et al., J. Biol. Chem. 260:1979-1982, 1985; Trere et al., Br. J. Cancer, 81: 404-8, 1999).

By “substantially pure” is meant a nucleic acid, polypeptide, or other molecule that has been separated from the components that naturally accompany it. Typically, the polypeptide is substantially pure when it is at least 60%, 70%, 80%, 90% 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications.

By “an effective amount” is meant an amount of a compound, alone or in a combination according to the invention, required to inhibit the growth of a neoplasm in vivo. The effective amount of active compound(s) used to practice the present invention for therapeutic treatment of neoplasms (i.e., cancer) varies depending upon the manner of administration, the age, body weight, and general health of the subject. Ultimately, the attending physician or veterinarian will decide the appropriate amount and dosage regimen. An effective amount of an asialo-interferon for the treatment of liver cancer is as little as 0.005, 0.01, 0.02, 0.025, 0.05, 0.075, 0.1, 0.133 mg per dose, or as much as 0.15, 0.399, 0.5, 0.57, 0.6, 0.7, 0.8, 1.0, 1.25, 1.5, 2.0 or 2.5 mg per dose. The dose may be administered once a day, once every two, three, four, seven, fourteen, or twenty-one days. The amount of the asialo-interferon administered to treat liver cancer is based on the asialo-interferon activity. It is an amount that is sufficient to effectively reduce cell proliferation or tumor size.

By “fragment” is meant a portion of a protein or nucleic acid that is substantially identical to a reference protein or nucleic acid, and retains at least 50%, 75%, 80%, 90%, or 95%, or even 99% of the biological activity (e.g., the anti-proliferative activity) of the reference protein or nucleic acid.

Other features and advantages of the invention will be apparent from the detailed description, and from the claims.

### BRIEF DESCRIPTION OF THE DRAWINGS

FIG. 1 is a schematic illustration of the structure of natural human interferon-β. Also illustrated are the cleavage sites of typical biantennary complex-type sugar chains of natural human interferon-β by neuraminidase. Abbreviations: Fuc, fucose; GIcNAc, N-acetylglucosamine; Man, mannose; Gal, galactose; NeuAc, N-acetylneuraminic acid (sialic acid).

FIG. 2A is the amino acid sequence of a human interferon-α-2 precursor polypeptide (Accession No.:P01563) (SEQ ID NO:1), including the signal peptide (amino acids 1-23; bold text). The mature interferon-α-2 polypeptide (plain text) extends from amino acid 24-188. The underlined threonine at amino acid 129 is the site of O-linked glycosylation.

FIG. 2B is the nucleic acid sequence (Accession No.: NM_{—}000605) (SEQ ID NO:4) of an mRNA that encodes human interferon-α-2 precursor polypeptide. The coding sequence extends from nucleic acid 69 to nucleic acid 635. The start and stop codons are underlined. Several variant forms of this nucleic acid sequence exist, which include the following nucleic acid changes: A to G at nucleic acid position 205; A to G at nucleic acid position 667; C to T at nucleic acid position 909; and/or A to G at nucleic acid position 949.

FIG. 3A is the amino acid sequence of a human interferon-β precursor polypeptide (Accession No.:P01574) (SEQ ID NO:2), including the signal peptide (amino acids 1-21; bold text). The mature human interferon-β polypeptide (plain text) extends from amino acid 22-187. The underlined asparagine at amino acid position 101 is the site of N-linked glycosylation. A human interferon-β variant polypeptide contains a tyrosine at amino acid position 162 (C to Y).

FIG. 3B is the nucleic acid sequence (Accession No:NM_{—}002176) (SEQ ID NO:5) of an mRNA that encodes human interferon-β precursor polypeptide. The coding sequence extends from nucleic acid 1-564. The start and stop codons are underlined. Several variant forms of this nucleic acid sequence exist, which include the following nucleic acid changes: C to T at nucleic acid position 153 and C to T at nucleic acid position 228.

FIG. 4A is the amino acid sequence of a human interferon-γ precursor protein (Accession No.:P01579) (SEQ ID NO:3) including the signal peptide (amino acids 1-20; bold text). The mature human interferon-γ polypeptide (plain text) extends from amino acid 21-166. The underlined asparagines at amino acid positions 48 and 120 of the interferon-γ precursor protein are the site of N-linked glycosylation (although Asn120 is only glycosylated in the dimer).

FIG. 4B is the nucleic acid sequence of an mRNA that encodes human interferon-γ precursor protein (NM_{—}000619) (SEQ ID NO:6). The coding sequence extends from nucleic acid 109-609. The start and stop codons are underlined. Several variant forms of this nucleic acid sequence exist, which include the following nucleic acid changes: A to G at nucleic acid 624; A to G at nucleic acid 705; A to T at nucleic acid 732; C to T at nucleic acid 789; C to T at nucleic acid 986; and A to G at nucleic acid 1148.

### DETAILED DESCRIPTION

Neoplastic hepatocytes frequently express the asialo-glycoprotein receptor and one or more interferon receptors. Asialo-interferon-α, -β, or -γ can be used to effectively treat liver cancer at dosages similar to or less than those used by those skilled in the art for the natural form of human interferon. Neoplastic hepatocytes contain two binding sites for asialo-interferon, the asialo-glycoprotein receptor and the interferon receptor. Equal or superior efficacy may be achieved at equivalent or lower doses of asialo-interferon as compared to native interferon; accordingly, toxicity and adverse side effects may be reduced.

Asialo-Glycoprotein Receptor

The asialo-glycoprotein receptor is a transmembrane protein, present at high density almost exclusively on hepatocytes (50,000-500,000 sites/cell), which mediates the binding and internalization of extracellular glycoproteins lacking terminal sialic acid residues. The asialo-glycoprotein receptor is a low affinity receptor, and it's affinity for ligand varies with the number of galactose clusters present on the ligand (Lee et al., J. Biol. Chem. 258:199-202, 1983). The receptor has a lower affinity for ligand having clusters of two galactose residues, biantennary (K_{D}˜10⁻⁶), than for ligand having clusters of three galactose residues, triantennary (K_{D}˜10⁻⁸ to 10⁻⁹).

Asialo-glycoprotein receptor expression is elevated in many hepatocellular carcinomas. Eisenberg et al. (J. Hepatol., 13: 305-309, 1991) have shown that while healthy livers have 140,000+/−65,000 asialo-glycoprotein binding sites per cell, the number of binding sites increases to 300,000+/−125,000 per cell in diseased livers having fibrosis, cirrhosis, or hepatocarcinoma (i.e., the receptor is “overexpressed”). Trere et al. have shown that eighty percent of well differentiated hepatocellular carcinomas (grade I and II cancers) and twenty percent of poorly differentiated hepatocellular carcinomas (grade III and IV) expressed asialo-glycoprotein receptors on their plasma membranes. Methods for identifying the presence of the asialo-glycoprotein receptor on cancer cells are well known to the skilled artisan (e.g., Hyodo et al., Liver 13:80-5,1993 Trere et al., Br. J. Cancer 81:404-8, 1999). A method for the non-invasive functional mapping of regional liver asialoglycoprotein receptor amount by single photon emission coaxial tomography is described by Shuke et al., J. Nucl. Med. 44:475-82, 2003.

Hepatic Delivery of Interferons

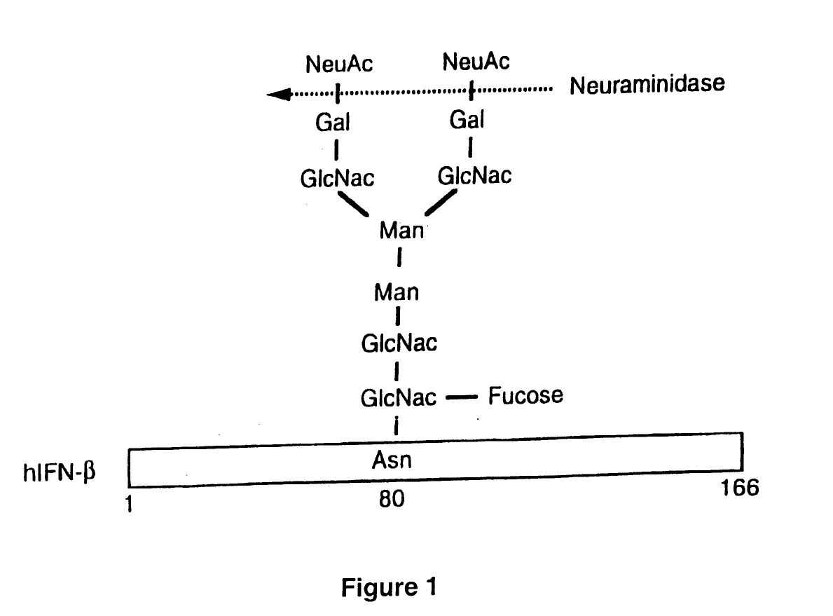

Removing a sialic acid group from any native interferon exposes the terminal galactose residues (FIG. 1), creating a recognition site for the asialo-glycoprotein receptor, and allowing the selective targeting of asialo-interferons to hepatocytes. This is particularly useful because the number of asialo-glycoprotein receptor binding sites increases in the diseased liver. Removal of the sialic acid group imparts several important therapeutic benefits to asialo-interferons, which makes them superior to native interferons. First, asialo-interferon is selectively targeted to the liver. Second, asialo-interferon is smaller than either native interferon or conjugated interferon and thus penetrates the liver fenestrae more effectively. Third, binding to the asialo-glycoprotein receptor and receptor complex internalization likely increases asialo-interferon's ability to activate intracellular interferon receptor pools. Finally, targeting asialo-interferon to the asialo-glycoprotein receptor likely increases the local concentration of asialo-interferon at the cell surface thus increasing the probability that asialo-interferon will bind an interferon receptor.

Cell Surface Interferon Receptor Binding

Increasing the local concentration of asialo-interferon on the hepatocyte surface, through binding to the asialo-glycoprotein receptor, increases the hepatic residence time and the probability that asialo-interferon-α, -β, or -γ will interact with an interferon receptor α/β or interferon γ receptor. High affinity interferon-α/β receptors (K_{D}˜10⁻¹²10⁻³¹), for example, are present on hepatocytes at low density (100-5,000 sites/cell). Because the asialo-glycoprotein receptor has a lower affinity for asialo-interferons than the interferon receptor, asialo-interferons are efficiently transferred from the abundant asialo-glycoprotein receptor to the less abundant interferon receptor. The affinity of the asialo-glycoprotein receptor for ligand varies with the number of galactose clusters present on its ligand (Lee et al., J. Biol. Chem. 258:199-202, 1983).

The asialo-glycoprotein receptor has a lower affinity for biantennary ligand (K_{D}˜10⁻⁶), than for triantennary ligand (K_{D}˜10⁻⁸ to 10⁻⁹). The extended conformation of the carbohydrate chain of interferon-β, for example, (Karpusas et al., Proc. Natl. Acad. Sci 94:11813-11818, 1997), likely permits simultaneous interaction with both the asialo-glycoprotein receptor and the interferon-α/β receptor. Thus, the abundant asialo-glycoprotein receptor may concentrate asialo-interferon-β at the cell surface where it likely interacts simultaneously with the less abundant interferon-α/β receptor.

Intracellular Interferon Receptor Binding

Binding of interferon-α, -β, or -γ to intracellular interferon receptors likely triggers interferon signaling. Interferon-α incorporated into liposomes can produce significantly greater activity than free interferon-α, supporting the hypothesis that interferons do not need to reach the cell surface to exert activity. Furthermore, ligand binding to the asialo-glycoprotein receptor triggers internalization of the receptor-ligand complex, providing asialo-interferons with access to intracellular interferon receptors.

Interferon Production

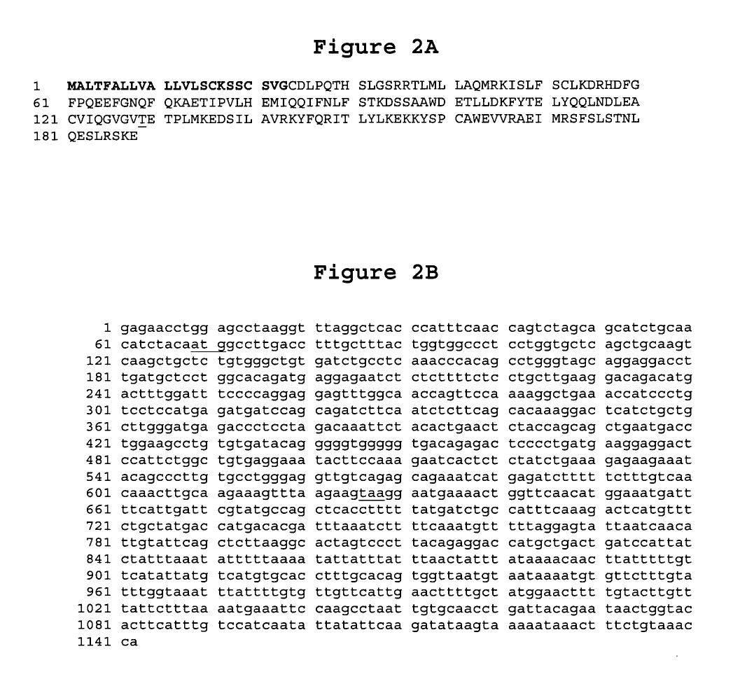

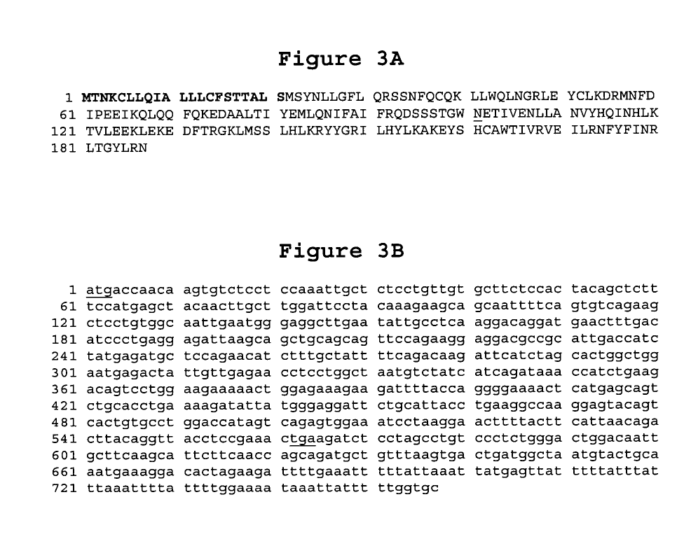

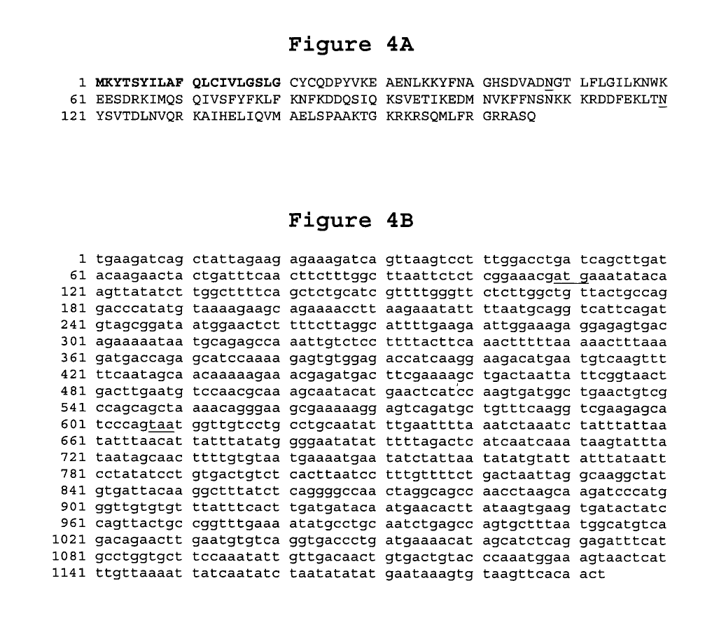

In general, polypeptides of the invention, such as interferon-α (FIG. 2A), -β (FIG. 3A), or -γ (FIG. 4A) may be produced by transformation of a suitable host cell, for example, a eukaryotic cell, with all or part of a polypeptide-encoding nucleic acid molecule, such as the interferon-α encoding nucleic acid shown in FIG. 2B, the interferon-β encoding nucleic acid shown in FIG. 3B, the interferon-γ encoding nucleic acid shown in FIG. 4B or a fragment thereof in a suitable expression vehicle.

Those skilled in the field of molecular biology will understand that any of a wide variety of expression systems may be used to provide the recombinant protein. Eukaryotic interferon peptide expression systems may be generated in which an interferon peptide gene sequence is introduced into a plasmid or other vector, which is then used to transform living cells. Constructs in which the interferon peptide cDNA contains the entire open reading frame inserted in the correct orientation into an expression plasmid may be used for protein expression. Eukaryotic expression systems allow for the expression and recovery of interferon peptide fusion proteins in which the interferon peptide is covalently linked to a tag molecule which facilitates identification and/or purification. An enzymatic or chemical cleavage site can be engineered between the interferon peptide and the tag molecule so that the tag can be removed following purification.

Typical expression vectors contain promoters that direct the synthesis of large amounts of mRNA corresponding to the inserted interferon peptide nucleic acid in the plasmid-bearing cells. They may also include an origin of replication sequence allowing for their autonomous replication within the host organism, sequences that encode genetic traits that allow vector-containing cells to be selected for in the presence of otherwise toxic asialo-interferons, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

The precise host cell used is not critical to the invention. A polypeptide of the invention may be produced in any eukaryotic host (e.g., Saccharomyces cerevisiae, insect cells, such as Sf21 cells, or mammalian cells, such as NIH 3T3, HeLa, COS cells, or fibroblasts). Such cells are available from a wide range of sources (e.g., the American Type Culture Collection, Rockland, Md.; also, see, e.g., Ausubel et al., Current Protocols in Molecular Biology, Wiley Interscience, New York, 2001). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

Native, glycosylated interferon can be isolated from human cells, which produce it naturally, or from transgenic eukaryotic cells that have been engineered to express a recombinant interferon gene. Methods for natural or recombinant production of interferon are generally described in U.S. Pat. Nos. 4,758,510, 4,124,702, 5,827,694, 4,680,261, 5,795,779, 5,376,567, and 4,130,641.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). The recombinant protein can be purified by any appropriate techniques, including, for example, high performance liquid chromatography chromatography or other chromatographies (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein). Alternatively, isolated and purified human interferon is available commercially (e.g., Sigma Chemical Co. as catalog Nos.: I 2396, I 2271, I 1640, and I 6507.

Preparation of Asialo-Interferon

Various methods are known for creating interferons having differing proportions of biantennary complexes. Interferons produced by fibroblast cells, for example, have a higher proportion of biantennary complexes than interferons produced by Chinese hamster ovary (CHO) cells. Specifically, human asialo-interferon-β produced in human fibroblasts contains about 82% biantennary galactose-terminal oligosaccharides and about 18% triantennary galactose-terminal oligosaccharides.

Asialo-interferon can be produced by removing a terminal sialic residue from interferon which is glycosylated by virtue of production in a eukaryotic cell (see, e.g., U.S. Pat. No. 4,184,917 and references cited therein, and Kasama et al., J. Interfer. Cyto. Res. 15:407-415, 1995). The terminal sialic residue can be removed, for example, by mild acid hydrolysis or treatment of native glycosylated interferon with isolated and purified bacterial or viral neuraminidase as described in Drzenieck et al., Microbiol. Immunol. 59:35, 1972. Neuraminidases are readily available from Sigma Chemical Co. (St. Louis, Mo.) (Catalog Nos. N 3642, N 5146, N 7771, N 5271, N 6514, N 7885, N 2876, N 2904, N 3001, N 5631, N 2133, N 6021, N 5254, and N 4883). Other methods of producing asialo-interferons are generally described in U.S. Pat. No. 6,296,844 (hereby incorporated by reference).

For instance, to produce human asialo-interferon-β, 20 mg of insoluble neuraminidase attached to beaded agarose (about 0.22 units) may be suspended in 1 ml distilled water in a microcentrifuge tube and allowed to hydrate briefly. The agarose may be pelleted by centrifugation and washed three times with 1 ml of sodium acetate buffer (pH 5.5) containing 154 mM NaCl and 9 mM calcium chloride and the gel (about 72 μl) may be re-suspended in 150 μl of the sodium acetate buffer. For example, glycosylated human interferon-β (3×10⁶ IU/vial, about 0.15 mg) may be suspended in 150 μl of sodium acetate buffer. The gel and interferon-β can then mixed and incubated on a rotating platform at 37° C. for three hours and the mixture can be separated from the neuraminidase by centrifugal filtration through a 0.2 μm filter. The asialo-interferon may be stored at −80° C. for extended periods of time.

A further exemplary method of preparing asialo-interferon involves digesting natural human interferon-β with one unit of Arthrobacter ureafaciens-derived neuraminidase in 1 ml of 5 mM formic acid (pH 3.5) at 37° C. for three hours. Following hydrolysis, the desialylated interferon-β may be isolated on a C18 reversed-phase column (e.g., Zorbax® PR-10) with a linear gradient of acetonitrile in 0.1% trifluoroacetic acid. Other methods of producing asialo-interferons are generally described in U.S. Pat. No. 6,296,844 (hereby incorporated by reference).

Formulation of Pharmaceutical Compositions

The administration of an asialo-interferon compound may be by any suitable means that results in a concentration of the asialo-interferon that, combined with other components, is anti-neoplastic upon reaching the target region. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the asialo-interferon within the body over an extended period of time; (ii) formulations that after a predetermined lag time create a substantially constant concentration of the asialo-interferon within the body over an extended period of time; (iii) formulations that sustain asialo-interferon action during a predetermined time period by maintaining a relatively, constant, effective asialo-interferon level in the body with concomitant minimization of undesirable side effects associated with fluctuations in the plasma level of the active asialo-interferon substance (sawtooth kinetic pattern); (iv) formulations that localize asialo-interferon action by, e.g., spatial placement of a controlled release composition adjacent to or in the diseased tissue or organ; (v) formulations that allow for convenient dosing, such that doses are administered, for example, once every one or two weeks; and (vi) formulations that target asialo-interferon action by using carriers or chemical derivatives to deliver the asialo-interferon to a particular target cell type. Administration of asialo-interferon compounds in the form of a controlled release formulation is especially preferred for asialo-interferons having a narrow absorption window in the gastro-intestinal tract or a very short biological half-life. In these cases, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the asialo-interferon is formulated with appropriate excipients into a pharmaceutical composition that, upon administration, releases the asialo-interferon in a controlled manner. Examples include single or multiple unit tablet or capsule compositions, oil solutions, suspensions, emulsions, microcapsules, microspheres, molecular complexes, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active asialo-interferon(s), the composition may include suitable parenterally acceptable carriers and/or excipients. The active asialo-interferon(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in the form suitable for sterile injection. To prepare such a composition, the suitable active asialo-interferon(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active asialo-interferon(s) may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Solid Dosage Forms For Oral Use

Formulations for oral use of interferon include tablets containing the active ingredient(s) in a mixture with non-toxic pharmaceutically acceptable excipients. Such formulations are known to the skilled artisan (e.g., 5,824,300, 5,817,307, 5,830,456, 5,846,526, 5,882,640, 5,910,304, 6,036,949, 6,036,949, 6,372,218, hereby incorporated by reference). Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active asialo-interferon substance in a predetermined pattern (e.g., in order to achieve a controlled release formulation) or it may be adapted not to release the active asialo-interferon substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active asialo-interferon substance). The coating may be applied on the solid dosage form in a similar manner as that described in Encyclopedia of Pharmaceutical Technology, supra.

The two asialo-interferons may be mixed together in the tablet, or may be partitioned. In one example, the first asialo-interferon is contained on the inside of the tablet, and the second asialo-interferon is on the outside, such that a substantial portion of the second asialo-interferon is released prior to the release of the first asialo-interferon.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active asialo-interferon by controlling the dissolution and/or the diffusion of the active asialo-interferon substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the asialo-interferon(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Combination Therapies

Asialo-inteferons may be administered in combination with any other standard cancer therapy; such methods are known to the skilled artisan (e.g., Wadler et al., Cancer Res. 50:3473-86, 1990), and include, but are not limited to, chemotherapy, radiotherapy, and any other therapeutic method used for the treatment of cancer.

### EXAMPLE I

To produce human asialo-interferon-β, 20 mg of insoluble neuraminidase attached to beaded agarose (about 0.22 units) is suspended in 1 ml distilled water in a microcentrifuge tube and allowed to hydrate briefly. The agarose is pelleted by centrifugation and washed three times with 1 ml of sodium acetate buffer (pH 5.5) containing 154 mM NaCl and 9 mM calcium chloride. The gel (about 72 μl) is resuspended in 150 μl of the sodium acetate buffer. Glycosylated human interferon-β (3×10⁶ IU/vial; about 0.15 mg) is suspended in 150 μl of sodium acetate buffer. The gel and interferon-β are then mixed and incubated on a rotating platform at 37° C. for three hours. The mixture is separated from the neuraminidase by centrifugal filtration through a 0.2 gm filter. The asialo-interferon may be stored at −80° C. for extended periods of time.

The method described above can also be used to prepare the asialo forms of interferon-α and interferon-γ. Other methods for preparing asialo glycoproteins are also widely known, for example, acid hydrolysis (e.g., Duk et al., Glycoconj J. 9:148-53, 1992).

Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treating a patient having liver cancer, said method comprising administering to said patient an effective amount of a pharmaceutical composition comprising a mammalian asialo-interferon.

2. The method of claim 1, wherein said liver cancer expresses the asialo-glycoprotein receptor.

3. The method of claim 2, wherein said liver cancer overexpresses the asialo-glycoprotein receptor.

4. The method of claim 1, wherein said liver cancer is selected from the group consisting of diffuse-type hepatocellular carcinoma, febrile-type hepatocellular carcinoma, cholestatic hepatocellular carcinoma, hepatoblastoma, hepatoid adenocarcinoma, and focal nodular hyperplasia.

5. The method of claim 1, wherein said asialo-interferon is a human asialo-interferon.

6. The method of claim 5, wherein said human asialo-interferon is an asialo-interferon-α.

7. The method of claim 5, wherein said human asialo-interferon is an asialo-interferon-β or an asialo-interferon-γ.

8. The method of claim 1, wherein said effective amount is 0.05-1.5 mg/week.

9. The method of claim 1, wherein said method further comprises a second anti-neoplastic therapy.

10. The method of claim 9, wherein said second anti-neoplastic therapy is chemotherapy or radiation therapy.

11. A method for treating a patient having liver cancer, said method comprising the steps of:
(a) testing said liver cancer for expression of an asialo-glycoprotein receptor; and
(b) if said testing step (a) is indicative that said liver cancer expresses an asialo-glycoprotein receptor, administering to said patient an effective amount of a composition comprising a mammalian asialo-interferon.

12. The method of claim 11, wherein said testing in step (a) comprises performing a liver biopsy.

13. The method of claim 11, wherein said liver cancer overexpresses the asialo-glycoprotein receptor.

14. The method of claim 13, wherein said testing in step (a) comprises non-invasive imaging the liver of said patient.

15. The method of claim 11, wherein said liver cancer is selected from the group consisting of diffuse-type hepatocellular carcinoma, febrile-type hepatocellular carcinoma, cholestatic hepatocellular carcinoma, hepatoblastoma, hepatoid adenocarcinoma, and focal nodular hyperplasia.

16. The method of claim 11, wherein said asialo-interferon is a human asialo-interferon.

17. The method of claim 16, wherein said human asialo-interferon is asialo-interferon-α.

18. The method of claim 16, wherein said human asialo-interferon is asialo-interferon-β or asialo-interferon-γ.

19. The method of claim 11, wherein said effective amount is 0.05-1.5 mg/week.

20. The method of claim 11, wherein said method further comprises a second anti-neoplastic therapy.

21. A method for treating a metastatic cancer of the liver, said method comprising administering to said patient an effective amount of a pharmaceutical composition comprising a mammalian asialo-interferon.

22. The method of claim 21, wherein said metastatic cancer is selected from the group consisting of metastatic prostate cancer, metastatic colorectal cancer, metastatic breast cancer, metastatic lung cancer, metastatic pancreatic cancer, metastatic melanoma, metastatic leukemia, and metastatic lymphoma.

23. The method of claim 21, wherein said human asialo-interferon is asialo-interferon-α, asialo-interferon-β or asialo-interferon-γ.
